(19) United States
(12) Patent Application Publication
(10) Pub. No.: US 2024/0130952 A1
(43) Pub. Date: Apr. 25, 2024

(54) REACTIVE TOPICAL SKIN PROTECTANT FORMULATIONS FOR THE PROTECTION AGAINST SKIN PENETRATION OF LIQUID CHEMICAL AGENTS OR HARMFUL TOXIC SUBSTANCES

(71) Applicant: AGENCY FOR DEFENSE DEVELOPEMENT, Daejeon (KR)

(72) Inventors: Hae Eun JOE, Daejeon (KR); Chang Hwan KIM, Daejeon (KR); Dong Sung SEO, Daejeon (KR); Sang Woong KIM, Daejeon (KR); Young CHO, Daejeon (KR)

(21) Appl. No.: 18/127,391

(22) Filed: Mar. 28, 2023

(30) Foreign Application Priority Data
Mar. 29, 2022 (KR) 10-2022-0038712

(51) Int. Cl. A61K 8/81; A61K 8/19; A61K 8/34; A61K 8/70; A61K 8/73; A61K 8/891; A61Q 17/00

(52) U.S. Cl. CPC A61K 8/8123; A61K 8/19; A61K 8/345; A61K 8/70; A61K 8/733; A61K 8/891; A61Q 17/00

(57) ABSTRACT

Provided are the reactive topical skin protectant formulations including: water or moisture; plate-like inorganic particle powder; and one or more selected from the group consisting of a) liquid perfluoride, b) polyol, c) perfluorinated polymer powder, d) polysaccharides, e) liquid silicon-based material, and f) additives, and the reactive topical skin protectant formulations may provide an effect of safely protecting the skin from various lesions and risks occurring by liquid chemical agents or harmful toxic substances penetrating the human skin.

### CROSS-REFERENCE TO RELATED APPLICATIONS

This application claims priority under 35 U.S.C. § 119 to Korean Patent Application No. 10-2022-0038712, filed on Mar. 29, 2022, in the Korean Intellectual Property Office, the disclosure of which is incorporated herein by reference in its entirety.

### TECHNICAL FIELD

The following disclosure relates to reactive topical skin protectant formulations for the protection against skin penetration of liquid chemical agents or harmful toxic substances.

### BACKGROUND

Among the chemical agents used in chemical, biological, and radiological situations and for terrorist purposes, nerve agents inhibit the action of enzyme acetylcholinesterase in nerve synapses to accumulate acetylcholine when it is absorbed into the body through the respiratory tract, digestive tract, and skin, so that symptoms such as contraction of pupils, difficulty in breathing, and muscle spasms are shown. In addition, when vesicating agents are also absorbed through the respiratory tract, digestive tract, and skin, they bind to biomolecules in the body to cause gene mutations or cancer, and react with various intracellular proteins to inhibit the action of enzymes and impair metabolism to destroy body tissue, such as inflammation and blistering.

In the case of exposure to these liquid chemical agents, even very small amounts may cause death, and thus, the development of various defense methods of blocking their penetration and absorption into the body has progressed.

The first method is to cover the skin with protective clothing made of non-woven fabric, films, fabric, rubber, and the like. Though body-friendly and lightweight protective clothing has been recently developed, the defense method has a problem of being unfavorable to protection of ears, nape, wrists and ankles, and groin which are vulnerable areas where their skin is highly likely to be exposed, the waist part where the top and the bottom are connected, and the like.

A second method attempted for solving the problem is a defense method of applying a material to protect the skin against chemical agents, but the defense method also has a limitation like the defense method of using protective clothing. For example, an active material which itself may be harmful may be applied on the skin for incapacitating the chemical agents, and one should endure it for a long time with a sticky and unpleasant sensation before and after application, nevertheless the defensive effect is still limited and insufficient. In addition, a defense method using the form of forming a film by applying the formulations including an active material to the skin is not effective in defense against a chemical agent, contrary to expectation. The defense method of forming a film may cause cracks on the film as time passed after application due to human body motion, and the skin is exposed beneath the cracked film and the chemical agent may penetrate there.

Accordingly, attempts to develop a skin application defense method against a liquid chemical agent to replace a conventional defense method against skin damage occurring when a chemical agent sprayed by someone comes into contact with the soldier's or civilian's skin in situations of war or terrorism continued, and are, for example, as follows.

In the United States, a skin exposure protective paste against chemical warfare agent (SERPACWA) including a fluorine-based oil and fluorine-based polymer powder as a protectant for protecting the soldier's skin in chemical agent-exposed mission oriented protective posture (MOPP) situations was developed. However, since the developed SERPACWA has no detoxification efficacy to neutralize the toxicity of the chemical agent, it was limited to having only a protective role. Thereafter, in order to complement the protective efficacy of SERPACWA, a reactive topical skin protectant (rTSP) having detoxification activity by further newly including amine or polyalkenimine (U.S. Pat. No. 7,976,832 B2) or including polyoxometalate (U.S. Pat. No. 6,420,434 B1) was developed, but was not able to be put into practical use and distributed to the military.

In Israel, a formulation including glycerin to react with thio compounds, oxidants, transdermal compounds, and various additives, (Korean Patent Registration Publication No. 10-0853443 B1) was developed. It has been known that when the formulation is applied on the skin, it is absorbed into the stratum corneum and a defensive effect against a sulfur mustard gas, which is a blistering agent and nerve agents, lasts for about 12 hours. This also had some degree of effect on the gaseous blistering agent, but did not secure sufficient protection against various chemical agents, in particular, liquid chemical agents.

Eventually, in spite of numerous efforts to solve the previous problems of a protective method against liquid chemical agents or harmful toxic substances, the technology in this area has not provided a satisfactory defense method for skin protection to provide a sufficient solution to the problem or overcome the disadvantages so far.

### RELATED ART DOCUMENTS

### Patent Documents

U.S. Pat. No. 7,976,832 B2

U.S. Pat. No. 6,420,434 B1

Korean Patent Registration Publication No. 10-0853443 B1

### Non-Patent Documents

Ernest H. Braue, Jr. “Development of an active topical skin protectant”, USAMRICD-TR-16-03 (pp. 1-180, 2016. 2.)

### SUMMARY

An embodiment of the present invention is directed to providing a new defense method for protecting the skin by performing chemical detoxification as well as blocking penetration of chemical agents or harmful toxic substances through the skin by a physical method by developing a skin protectant in the form which may be applied on the skin in advance, when there is a possibility of being exposed various kinds of liquid chemical agents or harmful toxic substances.

In one general aspect, reactive topical skin protectant formulations include: water or moisture; plate-like inorganic particle powder; and one or more selected from the group consisting of a) liquid perfluoride, b) polyol, c) perfluorinated polymer powder, d) polysaccharide, e) liquid silicon-based material, and f) additives.

In an exemplary embodiment of the present disclosure, the water or moisture may be included at 0.001 to 2 wt % based on the total weight of the reactive topical skin protectant formulations.

In an exemplary embodiment of the present disclosure, the plate-like inorganic particle powder may be included at 0.1 to 30 wt % based on the total weight of the reactive topical skin protectant formulations.

In an exemplary embodiment of the present disclosure, the moisture may be included at 0.1 to 5 parts by weight based on 100 parts by weight of the plate-like inorganic particle powder.

In an exemplary embodiment of the present disclosure, the plate-like inorganic particle powder may have an average particle diameter of 0.1 to 10 μm.

In an exemplary embodiment of the present disclosure, the plate-like inorganic particle powder may be one or more selected from mica, pearl, talc, bentonite, boron nitride, graphite, and aluminum powder pressed thinly with a roller.

In an exemplary embodiment of the present disclosure, the plate-like inorganic particle powder may be surface-treated with one or more inorganic substances selected from magnesium oxide, magnesium hydroxide, magnesium carbonate, aluminum oxide, aluminum hydroxide, silica, hydrated silica, calcium oxide, calcium hydroxide, calcium carbonate, titanium dioxide, titanium hydroxide, iron oxide, iron hydroxide, zinc oxide, zinc hydroxide, zinc carbonate, zirconium oxide, zirconium hydroxide, cerium oxide, and cerium hydroxide.

In an exemplary embodiment of the present disclosure, the surface treatment may include dispersing the plate-like inorganic particle powder in a metallic salt aqueous solution; adding an alkaline aqueous solution to form precipitate in a range of pH 9 to 10 to perform aggregation; and performing heating at 400 to 500° C. for 30 minutes to 3 hours.

In an exemplary embodiment of the present disclosure, the inorganic substance may be included at 0.5 to 20 parts by weight based on 100 parts by weight of the plate-like inorganic particle powder.

In an exemplary embodiment of the present disclosure, 10 to 85 wt % of the liquid perfluoride, 5 to 60 wt % of the polyol, 5 to 60 wt % of the perfluorinated polymer powder, 0.001 to 2 wt % of the polysaccharide, and 0.01 to 10 wt % of the additives may be included based on the total weight of the reactive topical skin protectant formulations.

In an exemplary embodiment of the present disclosure, the perfluoride may be represented by the following Structural Formula 1:

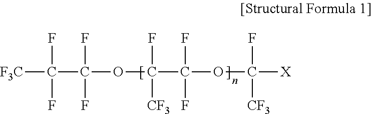

wherein n is an integer of 1 to 500, and X is selected from the group consisting of F, acyl fluoride, acrylate, C1-C4 alcohol, carboxylic acid, and methyl ester.

In an exemplary embodiment of the present disclosure, the perfluorinated polymer powder may have an average particle diameter of 1 to 10 μm and a specific surface area of 3 to 15 m²/g.

In an exemplary embodiment of the present disclosure, the liquid silicon-based material may be represented by the following Structural Formula 2:

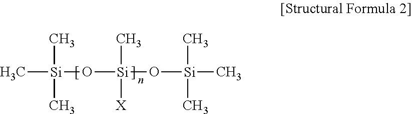

wherein n is an integer of 1 to 500, and X is hydrogen or a methyl group.

In an exemplary embodiment of the present disclosure, the additive may be one or more selected from a fluorine-based film forming agent, fluorine-based surfactant, fluorine-based thickener, fluorine-based solvent, silicon-based film forming agent, silicon-based surfactant, silicon-based thickener, lipid-based raw material, and an inorganic salt adjusting agent.

In an exemplary embodiment of the present disclosure, the reactive topical skin protectant formulations may be formulated into one or more selected from the group consisting of cream, ointment, gel, foundation, and stick.

In an exemplary embodiment of the present disclosure, the reactive topical skin protectant formulations may be for protecting the skin from lesions caused by transdermal penetration of liquid chemical agents or harmful toxic substances.

Other features and aspects will be apparent from the following detailed description, the drawings, and the claims.

### BRIEF DESCRIPTION OF THE DRAWINGS

FIG. 1 is a schematic diagram showing protection against skin penetration of liquid chemical agents or harmful toxic substances, in which (a) shows the prior art and (b) shows the reactive skin protectant formulation according to the present disclosure.

FIG. 2 shows an apparatus manufactured for observing a protective effect of the reactive skin protectant formulation using KM9 detection paper which shows color change when liquid chemical agents or harmful toxic substances penetrate, and a test method in order.

FIGS. 3A and 3B are electron microscope photographs of fine plate-like powders or plate-like composite powders in which the plate-like powder is surface-treated with a different kind of inorganic substance of the present disclosure.

FIG. 4 shows a UV-Vis spectrum in which the surface-treated plate-like composite powders of Preparation Examples 1 to 4 decomposed DMNP.

FIG. 5A shows the summary of the diffusion cell test method and FIG. 5B shows the structure of the diffusion cell according to Experimental Example 3 of the present disclosure.

FIG. 6 is a graph showing cumulative penetration amounts of 2-CEPS in the mouse skin measured by the diffusion cell test method according to Experimental Example 3 of the present disclosure.

FIG. 7 is a graph representing a cumulative penetration amount of Dichlorvos into the mouse skin measured by the diffusion cell test method according to Experimental Example 3 of the present disclosure.

### DETAILED DESCRIPTION OF EMBODIMENTS

Hereinafter, new reactive topical skin protectant formulation according to the present disclosure which may safely protect the skin from various lesions and risks caused by penetration of liquid chemical agents or harmful toxic substances into the human skin will be described in detail with reference to the accompanying drawings. The next drawings were provided as examples so that the spirit of the present invention can be sufficiently transferred to a person skilled in the art to which the present invention pertains. Therefore, the present invention is not limited to the drawings provided below but may be embodied in many different forms, and the drawings suggested below may be exaggerated in order to clear the spirit of the present invention. In addition, the reference numerals used herein denote the equivalent elements throughout this specification.

Technical terms and scientific terms used herein have the general meaning understood by those skilled in the art to which the present invention pertains unless otherwise defined, and a description for the known function and configuration which may unnecessarily obscure the gist of the present disclosure will be omitted in the following description and the accompanying drawings.

The singular form used in the present specification may be intended to also include a plural form, unless otherwise indicated in the context.

In addition, units used in the present specification without particular mention are based on weights, and as an example, a unit of % or ratio refers to a wt % or a weight ratio and wt % refers to wt % of any one component in a total formulation, unless otherwise defined.

In addition, the numerical range used in the present specification includes all values within the range including the lower limit and the upper limit, increments logically derived in a form and span in a defined range, all double limited values, and all possible combinations of the upper limit and the lower limit in the numerical range defined in different forms. Unless otherwise defined in the specification of the present disclosure, values which may be outside a numerical range due to experimental error or rounding of a value are also included in the defined numerical range.

In addition, the term “comprise” in the present specification is an open-ended description having a meaning equivalent to the term such as “is/are provided”, “contain”, “have”, or “is/are characterized”, and does not exclude elements, materials, or processes which are not further listed.

In addition, the term “substantially not including” in the present specification means that other elements, materials, or processes which are not listed together with specified elements, materials, or processes may be present in an amount or extent which does not have an unacceptably significant effect on at least one basic and novel technical idea of the invention.

A method of causing suffering or death to an organism has been developed in a variety of forms and means. The organism reaches a critical condition or dies depending on the damage intensity or the action path including a physical blow or a chemical attack means applied to the organism. As such, various poisons which are a chemical attack weapon against humans are referred to as a chemical agent. Since the chemical agent classified as a chemical weapon leaves serious injuries and aftereffects, it has been used for the purpose of taking away a soldier's fighting power or causing death.

The chemical agent may be classified into a toxic agent, an incapacitating agent causing a temporary suppression effect without endangering life, a firing agent used for creating light beams and flames, and the like. These chemical agents are classified into four types such as a nerve agent, a blistering agent, a choking agent, and a blood agent, and these agents may be used for chemical warfare or terrorism mainly in a gaseous or liquid state.

Meanwhile, harmful toxic substances include various lethal pesticides, and in particular, since the chemical agent is a substance subject to strict control which is licensed for use only for special purpose in the practical use, it is specified that liquid simulant agents used for evaluation or comparison in the present disclosure are included in the range of the harmful toxic substance.

A representative example using a harmful toxic substance similar to the chemical agent is an act of spraying pesticides for controlling pests or microorganisms in the agricultural field. A chemical warfare having the same concept as pesticide spraying is an act of spraying a chemical agent which is a kind of pesticide in a broad sense on a specific area in a variety of ways to incapacitate an unspecified number of opposing forces.

Meanwhile, among the chemical agents, since a nerve agent and a blistering agent are hydrophobic compounds having a relatively large value of distribution coefficient (K_{o/w}) to two kinds of immiscible solvents, that is, octanol as a hydrophobic material and water as a hydrophilic material, they have a characteristic of passing through the skin well. Therefore, in order to develop a reactive topical skin protectant which makes it difficult for a liquid chemical agent to penetrate the skin and has excellent skin protection performance with detoxification, it is necessary to consider the following conditions:
first, a difference in interfacial chemical physical properties between the liquid chemical agent and the skin protection is large, a physical or mechanical barrier function is imparted to extend a penetration pathway, a reaction site where detoxification of the penetrated liquid chemical agent may occur is provided, a defense spectrum to various liquid chemical agents is large, and the like.

The human skin is composed of an epidermis including a stratum corneum, a clear layer, a granular layer, a polar layer, and a basal layer, a dermis including a papillary layer and a reticular layer, and subcutaneous tissue. The skin is an external organ which acts as a barrier to keep the shape, protect an internal organ, and also defend from a substance penetrating from the outside. That is, the skin is an important barrier to separate the outside and the inside of a living body, but responds to temperature and fear to sweat or shrinks to give goose bumps, and is also a sensitive sense organ sensing touch and pressure.

The skin sweats out and blocks penetration of moisture, but has a function to exchange substances selectively, such as allowing some drugs to pass through in limited amounts and move inside. As such, it is important to develop a reactive topical skin protectant which may actively block penetration of liquid chemical agents by using the complex characteristics of the skin well.

Thus, the present disclosure provides reactive topical skin protectant formulations which protect the skin against liquid blisters and skin damage or lesions caused by nerve agents or harmful toxic substances in contact with the skin, by contriving a reactive skin protectant formulation including: a small amount of water or moisture; fine plate-like powder or plate-like composite powder in which the fine plate-like powder is surface-treated with a different kind of inorganic substance; and one or more selected from the group consisting of a) liquid perfluoride, b) polyol, c) perfluorinated polymer powder, d) polysaccharide, e) liquid silicon-based material, and f) additives.

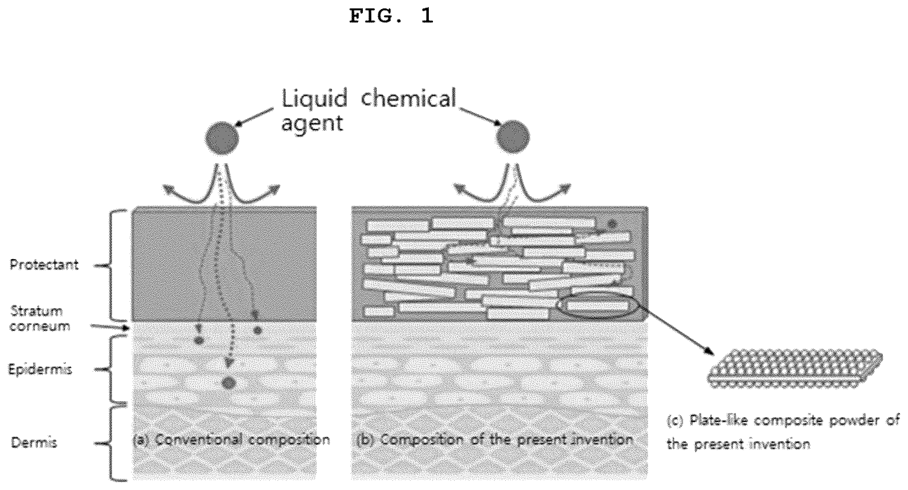

Comparing the action mechanism of the reactive topical skin protectant formulation according to the present disclosure shown in (b) of FIG. 1 with that of the prior art, the following is noted.

In terms of the fact that only when an interfacial tension difference with a liquid chemical agent or a harmful toxic substance is large, a skin protection effect is shown well, the two formulations according to (a) and (b) may be similar in that they are designed so that the liquid chemical agent or the harmful toxic substance rolls down from the applied skin surface or a contact area is so small that penetration into the skin is not easy. However, in the reactive skin protectant formulation (b) of the present disclosure, a physical or mechanical barrier is set and a function which extends the penetration path for bypass is newly imparted, plate-like composite powder (c) is applied for a detoxification function, and thus, an effect of effectively blocking the penetration of the liquid chemical agent or the harmful toxic substance into the skin may be provided.

The reactive topical skin protectant formulation of the present disclosure to which the new approach is applied has a more advanced performance of protecting the skin from the liquid chemical agents or the harmful toxic substances as compared with the prior art developed so far.

Hereinafter, each component of the reactive skin protectant formulation according to the present disclosure will be described in detail.

### A. Water or Moisture

Water or moisture is a core component to provide a reaction site where the reactive skin protectant formulation of the present disclosure may perform detoxification. In general, a process of removing or detoxifying liquid chemical agents or harmful toxic substances occurs by an adsorption or neutralization reaction. Here, the presence of water or moisture is important for the neutralization.

Though water and moisture are the same component having a chemical formula of H₂O, water and moisture in the present disclosure may be interpreted as the following definition. That is, water is the term used when a hydrophilic liquid component is combined with the formulation of the present disclosure, and moisture is the term used when a moisturizing process is performed for turning liquid water into a vapor state and introducing a hydroxyl group (—OH) or bound water acting as a reaction site for detoxification into fine pores of the plate-like composite powder according to the preparation example of the present disclosure.

In an exemplary embodiment of the present disclosure, the water or moisture may be included at 0.001 to 2 wt %, specifically 0.1 to 1.8 wt %, and more specifically 0.5 to 1.5 wt %, based on the total weight of the reactive skin protectant formulation. When it is included at 0.001 wt %, it is difficult to add water in a liquid state and a reaction site increase for detoxification as moisture is insignificant, which is thus not preferred. When it is included at more than 2.0 wt %, it is difficult to add it to the formulation and maintain a phase stably, which is thus not preferred also. Therefore, it is preferred that the water or moisture is included in the above range so that the reaction site for detoxification is appropriately secured and a stable phase is maintained.

It is preferred that moisture required for providing reaction sites for detoxification of the liquid chemical agent or the harmful toxic substance is present in fine pores of fine plate-like powder or plate-like composite powder in which the fine plate-like powder is surface-treated with a different kind of inorganic substance. Here, it is preferred that the content of moisture required is 0.1 to 5.0 parts by weight, specifically 1.0 to 3.0 parts by weight, based on 100 parts by weight of fine plate-like powder or plate-like composite powder in which the fine plate-like composite powder is surface-treated with a different kind of inorganic substance, in terms of the effect of providing a reaction site. When moisture is excessively contained in the fine pores of the plate-like composite powder, it may be condensed into liquid water, which is thus not preferred.

### B: Liquid Perfluoride

Perfluoride may be a perfluoropolyether (PFPE) compound represented by the following Structural Formula 1: It is preferred that the perfluoride is used in a range of 10 to 85 wt %, specifically 20 to 80 wt %, and more specifically 40 to 75 wt %, based on the total weight of the reactive skin protectant formulation.

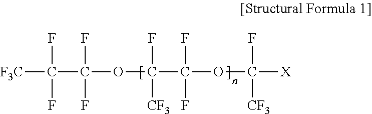

wherein n is an integer of 1 to 500, and X is selected from the group consisting of F, acyl fluoride, acrylate, C1-C4 alcohol, carboxylic acid, and methyl ester.

The specific compound represented by Structural Formula 1 is as follows:

### (Specific Compound 1-1) Perfluoropolyether Acyl Fluoride in which X is Substituted with Acyl Fluoride

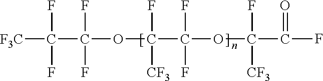

### (Specific Compound 1-2) Perfluoropolyether Acrylate in which X is Substituted with Acrylate

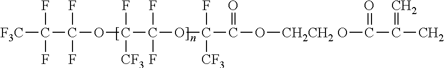

### (Specific Compound 1-3) Perfluoropolyether Alcohol in which x is Substituted with Alcohol

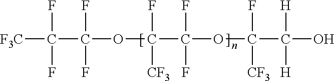

### (Specific Compound 1-4) Perfluoropolyether Carboxylic Acid in which X is Substituted with Carboxylic Acid

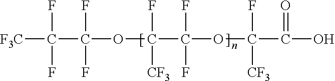

### (Specific Compound 1-5) Perfluoropolyether Methyl ester in which X is Substituted with Methyl Ester

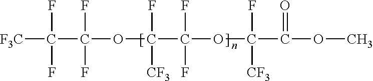

### C. Polyol

It is preferred that the polyol is included at 2 to 80 wt %, specifically 5 to 60 wt %, based on the total weight of the reactive topical skin protectant formulation of the present disclosure, since the skin protection effect is sufficient and appropriate viscosity is shown. Here, the polyol may be one or more selected from ethylene glycol, butylene glycol, propylene glycol, glycerin, diglycerin, and triglycerin, but is not limited thereto.

### D. Perfluorinated Polymer Powder

The perfluorinated polymer powder may be included in a range of 5 to 60 wt %, 5 to 30 wt %, or 5 to 20 wt %, based on the total weight of the reactive topical skin protectant formulation of the present disclosure. When the perfluorinated polymer powder is excessively used, the viscosity of the reactive topical skin protectant formulation of the present disclosure is too high as compared with an appropriate state and it is difficult to make a preparation such as cream in a continuous phase form, which is thus not preferred.

In addition, it is preferred to select polytetrafluoroethylene (PTFE) having an average particle size of 1.0 to 10.0 μm or 3.0 to 7.5 μm and a specific surface area in a range of 3.0 to 15.0 m²/g, as the powder.

### E. Plate-Like Inorganic Particles

The plate-like inorganic particles are specifically fine plate-like powder or a composite in which the fine plate-like powder is surface-treated with a different kind of inorganic substance, and are appropriately inorganic powder or metal powder in a thin and flat form like fish scales. The power may have an average particle size of 0.1 to 10.0 μm or 0.5 to 5 μm, and is preferably a thin layered compound. Specifically, for example, the inorganic particles may be one or more selected from mica, pearl, talc, bentonite, boron nitride, graphite, and aluminum powder pressed thinly with a roller.

In a preferred exemplary embodiment of the present disclosure, the plate-like inorganic particles may have significantly high detoxification activity by a surface treatment with a different kind of inorganic substance on the surface of the fine plate-like powder. The effect of the surface treatment is to increase a surface area on which the liquid chemical agent or the harmful toxic substance may be adsorbed, and provide hydroxyl groups (—OH) to the neutralization sites in a state where water or moisture is present, which is thus effective.

Thus, an appropriate different kind of inorganic substance may be one or more selected from magnesium oxide, magnesium hydroxide, magnesium carbonate, aluminum oxide, aluminum hydroxide, silica, hydrated silica, calcium oxide, calcium hydroxide, calcium carbonate, titanium dioxide, titanium hydroxide, iron oxide, iron hydroxide, zinc oxide, zinc hydroxide, zinc carbonate, zirconium oxide, zirconium hydroxide, cerium oxide, and cerium hydroxide. Here, it is preferred that the amount being surface-treated with a different kind of inorganic substance on the fine plate-like powder is 0.5 to 30 parts by weight or 1 to 20 parts by weight, based on 100 parts by weight of the fine plate-like powder. When it is less than 0.5 parts by weight, the effect of surface area increases and detoxification is insignificant, and when it is more than 30 parts by weight, the surface area is excessively increased to absorb the liquid added together to increase the viscosity of the preparation made into the formulation, which is thus not preferred.

A specific method of surface-treating the fine plate-like powder with a different kind of inorganic substance is as follows.

A divalent or higher water-soluble metallic salt is selected and dissolved in an aqueous phase, plate-like powder is dispersed, an alkaline aqueous solution is slowly added dropwise to the dispersion to cause aggregation as precipitation is formed in a range of pH 9 to 10 with continuous stirring, the aggregate is filtered, washed with water or alcohol, and dried, and then a heat treatment was performed at 400 to 500° C. for 1 hour.

Here, an appropriate divalent or higher water-soluble metallic salt is preferably a nitrate salt, a sulfate salt, and a hydrochloride salt. In addition, an alkaline aqueous solution added for forming a different kind of inorganic substance may be ammonia, sodium hydroxide, or potassium hydroxide aqueous solution.

As a preferred example of the present disclosure, combinations of the components described above may be A+B+E, A+C+E, A+B+D+E, or A+B+D. In this case, an excellent skin protection effect is shown, which is thus preferred.

### F. Polysaccharide

Since a polysaccharide may be hydrated to form a gel when being present with component A or C, it may act as a skin protection barrier for liquid chemical agents or harmful toxic substances. The polysaccharide may be included at 1 to 20 wt % or 2 to 10 wt % with respect to the total weight of the reactive topical skin protectant formulation of the present disclosure. When it is used at less than 1.0 wt %, a gel forming effect may not be expected, and when it is used at more than 20.0 wt %, a gel dries too quickly after application to the skin and the coated film may be cracked, which is thus not preferred.

The polysaccharide may be one or more selected from agar-agar, carrageenan, sodium alginate, guar gum, locust bean gum, xanthan gum, arabic gum, gellan gum, and konjac flour, but is not particularly limited thereto.

### G. Liquid silicon-based material

Liquid silicon-based material may be a compound represented by the following Structural Formula 2:

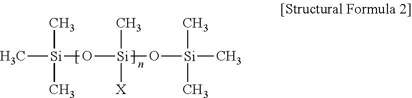

wherein n is an integer of 1 to 500 and represents a polymerization degree of a monomer [—OSi(CH₃)X—], and X is selected from hydrogen (—H) or a methyl group (—CH₃).

The liquid silicon-based material may be used as an auxiliary component which imparts an interfacial tension difference with hydrophobic liquid chemical agents or harmful toxic substances, and also, may impart a more comfortable feeling of use when applying the formulation of the present disclosure to the skin.

Specifically, the liquid silicon-based material may be included at 0.1 to 20 wt % or 0.5 to 10 wt % with respect to the total weight of the reactive topical skin protectant formulation. When it is used at less than 0.1 wt %, it is difficult to expect the effect of addition, and when it is used at more than 20.0 wt %, compatibility with other components may deteriorate, which is thus not preferred.

### H. Additives

Additives are added to a combination of essential components to provide various roles. The additive may be mainly a film forming agent, a surfactant, a thickener, a solvent, a lipid, or an inorganic salt adjusting agent, and may be included in a range of 0.01 to 10.0 wt % or 0.1 to 5 wt % with respect to the total weight of the reactive topical skin protectant formulation. When the amount of the additive used is less than 0.01 wt %, it is difficult to expect the effect of addition, and when it is added at more than 10.0 wt %, a skin protection effect deteriorates or it is not economical.

The additive may be specifically one or more selected from a fluorine-based film forming agent, a fluorine-based surfactant, a fluorine-based thickener, a fluorine-based solvent, a silicon-based film forming agent, a silicon-based surfactant, a silicon-based thickener, a lipid-based raw material, and an inorganic salt adjusting agent.

Specific components of the additive are suggested as follows.

The fluorine-based film forming agent may be one or more selected from a perfluoropropylene/vinylidene difluoride copolymer, polyperfluoroperhydrophenanthrene, an acrylates/perfluorohexylethyl methacrylate copolymer, C20-28 alkyl perfluorodecylethoxy dimethicone, a C6-14 perfluoroalkylethylacrylate/HEMA copolymer, a stearyl methacrylate/perfluorooctylethyl methacrylate copolymer, a PEG-10 acrylate/perfluorohexylethyl acrylate copolymer, and perfluorononylethyl stearyl dimethicone.

The fluorine-based surfactant may be one or more selected from TEA-C8-18 perfluoroalkylethyl phosphate, polyperfluoroethoxymethoxy PEG-2 phosphate, ammonium C9-10 perfluoroalkylsulfonate, perfluorononylethyl carboxydecyl PEG-10 dimethicone, behenyl methacrylate/perfluorooctylethyl methacrylate copolymer, perfluoromethyldecalin), perfluoromethylcyclohexane, perfluoroheptane, and ammonium C6-16 perfluoroalkylethyl phosphate.

The fluorine-based thickener may be one or more selected from C6-14 perfluoroalkylethyl acrylate/HEMA copolymer, a stearyl methacrylate/perfluorooctylethyl methacrylate copolymer, and polyperfluoroethoxymethoxy difluoromethyl distearamide.

The fluorine-based solvent may be one or more selected from perfluoroperhydrobenzyl tetralin, C6-12 perfluoroalkylethanol, perfluorocaprylyl bromide, perfluorodimethylcyclohexane, perfluoromethylcyclopentane, perfluorohexane, perfluoroperhydrophenanthrene, perfluorodecalin, ethyl perfluoroisobutyl ether, ethyl perfluorobutyl ether, methyl perfluoroisobutyl ether, and methyl perfluorobutyl ether.

The silicon-based film forming agent may be one or more selected from a silicone quaternium-16/glycidoxy dimethicone crosspolymer, polysilicone-6, polysilicone-9, polysilicone-14, polysilicone-11, polysilicone-8, and an acrylates/polytrimethylsiloxymethacrylate copolymer.

The silicon-based surfactant may be silicone quaternium-17.

The silicon-based thickener may be one or more selected from polysilicone-5, polysilicone-4, polysilicone-16,polysilicone-22), a polysilicone-1 crosspolymer, and polysilicone-17.

The lipid-based raw material may be specifically a phospholipid. For example, it may be one or more selected from phosphatidylcholine, phosphatidylserine, phosphatidylinositol, phosphatidylethanolamine, and sphingomyelin.

The inorganic salt adjusting agent may be one or more selected from lithium hydroxide (LiOH), sodium hydroxide (NaOH), potassium hydroxide (KOH), lithium carbonate (Li₂CO₃), sodium carbonate (Na₂CO₃), potassium carbonate (K₂CO₃), lithium hydrogen carbonate (LiHCO₃), sodium hydrogen carbonate (NaHCO₃), and potassium hydrogen carbonate (KHCO₃).

In addition, the present disclosure may provide a manufacturing method of a product variously formulated with the reactive topical skin protectant formulation. Various formulations in the form of cream, ointment, gel, foundation, and stick may be manufactured by including wetting the reactive topical skin protectant formulation described above, performing mixing, performing milling, performing outgassing, and performing aging. Thus, transdermal penetration of the liquid chemical agent or the harmful toxic substance in contact with the human skin may be prevented to protect the skin and also block occurrence of various lesions in advance.

Hereinafter, the present disclosure will be described in more detail by the following examples. However, the following examples are only a reference for describing the present invention in detail, and the present invention is not limited thereto and may be implemented in various forms.

### Preparation Example 1 Preparation of Boron Nitride Composite Surface-Treated with Nano-MgO Particles

5.13 g of magnesium nitrate (Mg(NO₃)₂·6H₂O) was dissolved in 200 mL of purified water, 20 g of plate-like boron nitride was added thereto, and dispersion was performed with a magnetic stirrer. 40 mL of 0.5 M sodium hydroxide (NaOH) was slowly added dropwise until pH reached 9 to 10, stirring was performed for 1 hour, and the white precipitate was filtered, washed three times with methanol and purified water, dried at 100° C. for 3 hours, and calcinated at 500° C. for 2 hours to obtain boron nitride composite powder surface-treated with nano-MgO particles.

### Preparation Example 2 Preparation of Mica Composite Surface-Treated with Nano-MgO Particles

The mica composite powder surface-treated with nano-MgO particles was obtained in the same manner as in Preparation Example 1, except that mica was used instead of boron nitride.

### Preparation Example 3 Preparation of Boron Nitride Composite Surface-Treated with Nano-CeO₂ Particles

8.68 g of cerium nitrate (Ce(NO₃)₂·6H₂O) was added to a flask containing 75 mL of ethanol, stirring was performed at room temperature for 2 hours, a mixed solution of 7.5 mL of ammonia water (NH₃·H₂O) and 7.5 mL of ethanol was slowly added dropwise, stirring was performed in a water bath at 50° C. for 2 hours, the product was washed with distilled water and ethanol 7 times, respectively, dried at 50° C. for 12 hours, and heated at 500° C. for 2 hours to obtain boron nitride composite powder surface-treated with nano-CeO₂ particles of the present disclosure.

### Preparation Example 4 Preparation of Mica Composite Surface-Treated with Nano-CeO2 Particles

The mica composite powder surface-treated with nano-CeO₂ particles was obtained in the same manner as in Preparation Example 3, except that mica was used instead of boron nitride.

### Examples 1 and 2

The reactive topical skin protectant formulations of the present disclosure were prepared by combining components shown in the following Table 1. Each component was mixed in order, and three-stage mill treatment was performed three times. The name of each component of the examples followed the rules of International Union of Pure and Applied chemistry (IUPAC) nomenclature for compounds or International Nomenclature Cosmetic Ingredients (INCI), and if necessary, the product name is indicated.

TABLE 1
Reactive topical skin protectant
formulations (unit: wt %)

| Component | | | |
|---|---|---|---|
| group | Component name | Example 1 | Example 2 |
| A | Water ¹⁾ | — | 3.0 |
| | Moisture | 1.3 ²⁾ | — |
| B | Perfluoropolyether | 60.0 | — |
| | Perfluoropolyether alcohol | 14.7 | — |
| C | Glycerin | — | 72.0 |
| D | Polytetrafluoroethylene | 6.0 | — |
| E | Plate-like boron nitride surface- | 18.0 | 18.0 |
| | treated with nano-MgO | | |
| F | Sodium alginate | — | 2.0 |
| H | Hydrogenated polydecene | — | 4.0 |
| | Acrylate/ | — | 1.0 |
| | polytrimethylsiloxymethacrylate | | |
| | copolymer | | |

| | | |
|---|---|---|
| Total | 100.0 | 100.0 |

¹⁾ Purified water (deionized water) was used.
²⁾ Hereinafter, Component E obtained in Preparation Example 1 was obtained by a humidifying treatment for 4 hours by supplying hot vapor from a steam generator in a closed device (oven) . After the humidifying treatment, a moisture content when measured with an infrared moisture meter was 1.3%.

### Comparative Examples 1 and 2

Cosmetic compositions having cream and liquid foundation formulations according to the compositions following the following Tables 2 and 3 were prepared and used as the comparative examples of a KM9 detection paper discoloration test for the reactive topical skin protectant formulation of the present disclosure.

TABLE 2
Cream cosmetic composition (unit: wt %)

| | Component name | Comparative |
|---|---|---|
| Classification | (INCI name) | Example 1 |
| Oily phase | Isostearyl alcohol | 1.5 |
| | Butylene glycol cocoate | 1.0 |
| | Ethylene cellulose | 0.5 |
| | Glyceryl stearate | 0.7 |
| | PEG-75 stearate | 0.5 |
| | Steareth-20 | 0.2 |
| | Octyldodecyl myristate | 5.0 |
| | Jojoba seed oil | 3.0 |
| | Dimethicone | 5.5 |
| | Caprylic/capric triglyceride | 3.0 |
| Aqueous phase | Purified water | 65.8 |
| | PEG-240/HDI Copolymer | 3.0 |
| | Bis-Decyltetradeceth-20 Ether | |
| | Glycerin | 8.0 |
| | Xanthan gum | 0.3 |
| | Ethylhexyl glycerin | 0.1 |
| | Phenoxyethanol | 0.9 |
| | Butylene glycol | 1.0 |
| | Cedar extract | 0.1 |

| | |
|---|---|
| Total | 100.0 |

The aqueous phase components of Table 2 were heated to 75° C. for 10 minutes, the oily components were slowly added thereto, stirring was performed at 3000 rpm, and the mixture was defoamed and cooled to room temperature with slow stirring to prepare the composition of Comparative Example 1.

TABLE 3
Composition of liquid foundation cosmetic
(unit: wt %)

| | Component name | Comparative |
|---|---|---|
| Classification | (INCI name) | Example 2 |
| Aqueous | 1,2-hexanediol | 1.0 |
| phase | Purified water | 41.5 |
| | Glycerin | 4.0 |
| | Dipropylene glycol | 4.0 |
| | Sodium chloride | 1.0 |
| Oily phase | Cyclopentasiloxane | 6.7 |
| | Ethylhexylpalmitate | 10.0 |
| | Trimethylsiloxysilicate | 1.0 |
| | Caprylic/capric triglyceride | 7.0 |
| | Bees wax | 2.0 |
| | Diisostearylmaleate | 2.5 |
| | Dimethicone | 1.0 |
| | Cetyl PEG/PPG-10/ | 4.2 |
| | 1-dimethicone | |
| | Sorbitan sesquioleate | 0.3 |
| | Distearyldimonium chloride | 0.2 |
| | Stearic acid | 0.3 |
| Solid phase | Disteadimonium hectorite | 1.3 |
| | Pigment base | 12.0 |
| | Total | 100.0 |

The aqueous components of Table 3 were dissolved at 78° C. The oily components were dissolved at 78° C. and dispersed at 4000 rpm for 5 minutes, and solid phase components were added in order and dispersed for 5 minutes. An aqueous phase was slowly added thereto, emulsification was performed for 12 minutes, and the product was defoamed and slowly cooled to room temperature to prepare the composition of Comparative Example 2.

### Examples 3 and 4

The reactive topical skin protectant formulation preparation of the present disclosure was prepared in the same manner as in Examples 1 and 2 by combining the component group shown in the following Table 4.

TABLE 4
Reactive topical skin
protectant formulations (unit: wt %)

| Component | | Example | Example |
|---|---|---|---|
| group | Component name | 3 | 4 |
| A | Purified water/moisture | 1.3 | 1.3 |
| B | Perfluoropolyether | 59.2 | — |
| | Perfluoropolyether alcohol | 15.0 | — |
| C | Glycerin | — | 72.7 |
| | Propylene glycol | — | 5.0 |
| D | Polytetrafluoroethylene | 20.0 | — |
| E | Boron nitride surface-treated | 4.5 | 15.0 |
| | with 25% nano-magnesium oxide | | |
| F | Sodium alginate | — | 1.0 |
| H | Hydrogenated polydecene | — | 4.0 |
| | Acrylate/ | — | 1.0 |
| | polytrimethylsiloxymethacrylate | | |
| | copolymer | | |

### Comparative Example 3

ICD #3280 of Table 1 of U.S. Pat. No. 6,420,434 B1 was selected as a comparative formulation.

### Comparative Example 4

Preparation E disclosed in KR 10-0853443 B which was originally filed in Israel was selected as a comparative formulation.

### Experimental Example 1. Discoloration Test Using KM9 Detection Paper

The skin protection performance of the formulations obtained in Examples 1 and 2 and Comparative Examples 1 and 2 were compared using a KM9 detection paper discoloration test.

KM9 detection paper is in a roll form of 5 cm wide and 9 cm long, which is attached to each soldier, equipment, or the like in chemical warfare to detect contamination with the liquid chemical agent by color change, and is special paper of which the color changes from green to red when it is in contact with the liquid chemical agent. It has a function to, when the color change is detected, protect individuals or equipment by wearing protective equipment or rapidly taking emergency measures. SG Safety Corporation, a domestic manufacture, produced M9 detection paper developed in the US and named it KM9 detection paper, which was purchased for use.

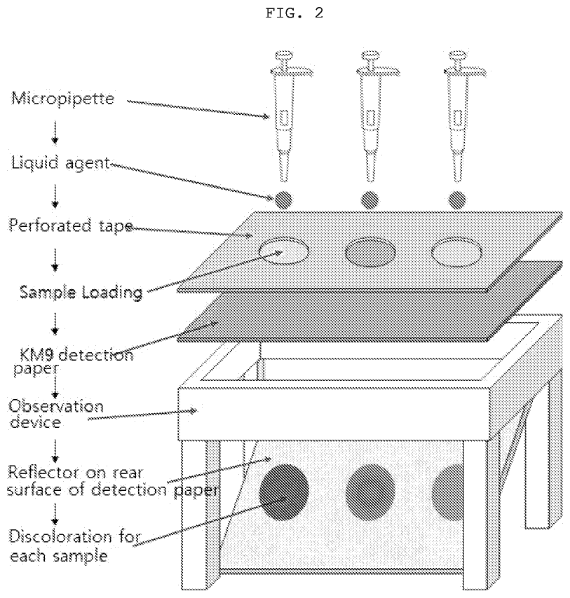

The present inventors manufactured an observation instrument as shown in FIG. 2 in order to perform the discoloration test of KM9 detection paper. The observation instrument was made of Teflon having excellent chemical resistance, and the discoloration of KM9 detection paper was visually observed through transparent glass. A reflector was installed at an angle of 45° in a lower portion of the device so that the discoloration was able to be confirmed on the rear surface (opposite side on which a comparative sample was applied) also of the KM9 detection paper when it was difficult to observe discoloration on the surface of the KM9 detection paper. The test method and the order were as shown in FIG. 2.

That is, a labeling tape perforated with round holes of a diameter of 16 mm at regular intervals was attached to the KM9 detection paper, which was fixed on the observation instrument of FIG. 2, and a round hole having an application area of 2 cm² was made on the label paper on the surface of the KM9 detection paper. About 100 μl of the samples (formulations) of the example of the comparative example in order to observe the discoloration was lightly added thereto with a pipette and evenly applied on the surface of the KM9 detection paper. The surface of the sample was removed any excess with slide glass to maintain a constant thickness of 0.25 mm. The sample was allowed to stand for about 30 minutes for stabilization and leveling of the sample layer of the example or the comparative example applied on the KM9 detection paper. 8 μl of dimethyl methyl phosphonate (DMMP) which is a liquid nerve agent simulant was lightly dropped on the applied surface of the sample with micropipette. Then, an onset time (time to start changing color) and discoloration strength were observed to see whether the KM9 detection paper was discolored over time. Whether colors of the KM9 detection paper with no formulation applied (control) and the KM9 detection paper on which the formulations of Examples 1 and 2 and Comparative Examples 1 and 2 were applied changed was observed immediately after dropping the formulation, after 5 minutes, after 10 minutes, after 15 minutes, after 30 minutes, after 60 minutes, and after 90 minutes in order, and finally change after 24 hours was observed.

The results of measuring the discoloration of the KM9 detection paper are shown in the Table 5.

The discoloration test results shown in Table 5 were only discoloration on the front and back surfaces of the KM9 detection paper immediately after dropping DMMP, after 5 minutes, after 60 minutes, and after 24 hours. For the rate at which the KM9 detection paper was discolored by penetrating DMMP into the applied sample, the control and the cream formulation of Comparative Example 1 having no protective effect against the liquid simulant agent completed all discoloration within 5 minutes, and the time to discoloration thereafter was a physical change due to absorption, diffusion, penetration, and sample drying of DMMP on the KM9 detection paper for the sample, and since the discoloration proceeded slowly, the discoloration difference of the KM9 detection paper between 5 minutes and 60 minutes was not significant. Therefore, the photographs of discoloration over time were those of 5 stages and are listed in Table 5.

According to the results of Table 5, it was shown that the formulations of Examples 1 and 2 of the present disclosure had excellent protective effect, and it was confirmed from the photographs of the surface (front surface) and the rear surface that the color of the KM9 detection paper by penetration or permeation of DMMP was not changed even after 24 hours. However, the cream formulation of Comparative Example 1 had almost no protective effect. The foundation formulation of Comparative Example 2 had a little protective effect, and foundation applied before 60 minutes was already penetrated by DMMP to cause discoloration, but the original color of the foundation was similar to the red color which occurred when the KM9 detection paper came into contact with DMMP, and thus, the discoloration was confirmed only after 60 minutes.

### Experimental Example 2. Detoxification Activity Evaluation

The detoxification activity of the plate-like composite powder according to the present disclosure was evaluated by the decomposition reaction of dimethyl p-nitrophenyl phosphate (DMNP) which is a simulant of the liquid chemical agent.

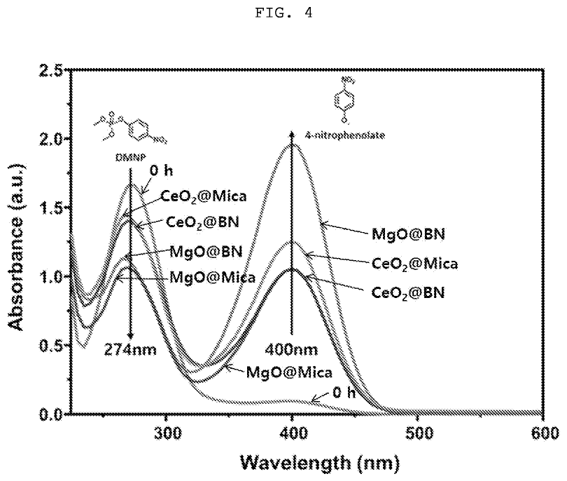

56 mg of the sample of the plate-like composite powder which was surface-treated with the nanoparticles obtained in Preparation Examples 1 to 4 and 1 mL of methanol were taken and added to a 4 mL vial, and ultrasonic dispersion was performed for 10 minutes. 5.6 mg (4 μl, 25 mmol) of DMNP was taken and added to the solution, stirring was performed at room temperature for 60 minutes, and the decomposition rate of DMNP and the production of Compounds 1 to 4 which were the decomposition products were measured by in situ UV-Vis spectroscopy and GC-MS. The results are shown in the following Table 6 and FIG. 4.

Table 6
Detoxification activity of plate-like
composite powder surface-treated with nanoparticles

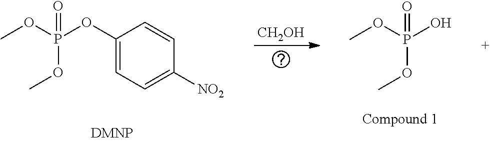

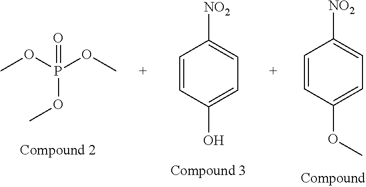

| | Detoxification | Decomposition product of DMNP | | | |
|---|---|---|---|---|---|
| Classification | rate (%) | Compound 1 | Compound 2 | \|Compound 3 | Compound 4 |
| Preparation | 66.9 | trace | 52.6 | 10.6 | 3.7 |
| Example 1 | | | | | |
| Preparation | 52.2 | trace | 37.8 | 10.5 | 3.9 |
| Example 2 | | | | | |
| Preparation | 33.3 | — | trace | 33.3 | — |
| Example 3 | | | | | |
| Preparation | 37.9 | trace | trace | 33.5 | 4.4 |
| Example 4 | | | | | |

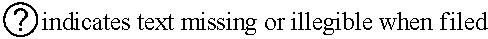

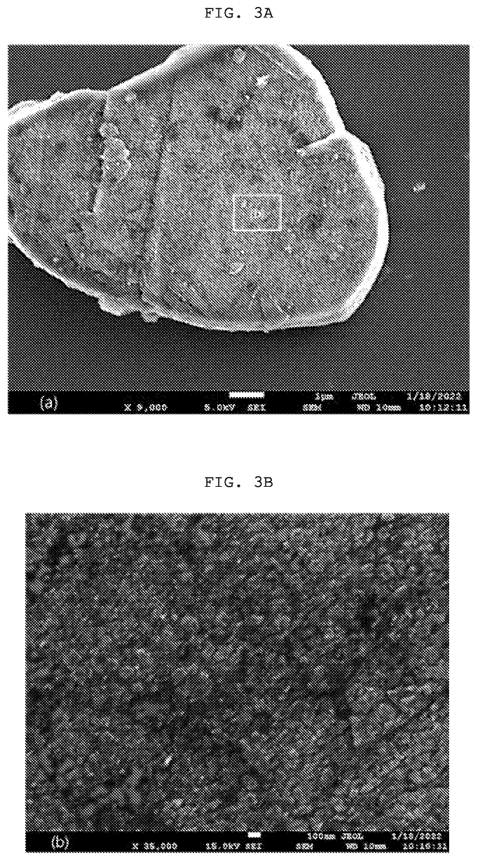

As shown in Table 6, the plate-like composite powders surface-treated with the nanoparticles according to the preparation examples of the present disclosure all showed detoxification activity, and in particular, boron nitride surface-treated with nano-MgO particles obtained in Preparation Example 1 exhibited excellent detoxification activity. It was considered that the detoxification activity of boron nitride surface-treated with nano-MgO particles of Preparation Example 1 was from the detoxification activity of the nanoparticles forming a composite on the surface of thin plate-like powder which was the characteristic structure of Component E of the present disclosure, as observed by the electron microscope shown in FIG. 3.

In addition, spectra in which the plate-like composite powder surface-treated with the nanoparticles according to the preparation examples of the present disclosure detoxified DMNP to produce Compound 4 as a decomposition product, as measured with UV-Vis spectroscopy, are shown in FIG. 4. As shown in FIG. 4, the plate-like composite powders surface-treated with the nanoparticles obtained in Preparation Examples 1 to 4, respectively, decomposed DMNP (λₘₐₓ=274 nm) to produce Compound 4 (4-nitrophenolate, λₘₐₓ=400 nm).

### Experimental Example 3. Diffusion Cell Penetration Test Using Mouse Skin

The detoxification and the skin protection efficacies of the formulations against the liquid chemical agent or the harmful toxic substance were measured as follows, by the diffusion cell penetration test using the mouse skin, for Examples 3 and 4 and Comparative Examples 3 and 4.

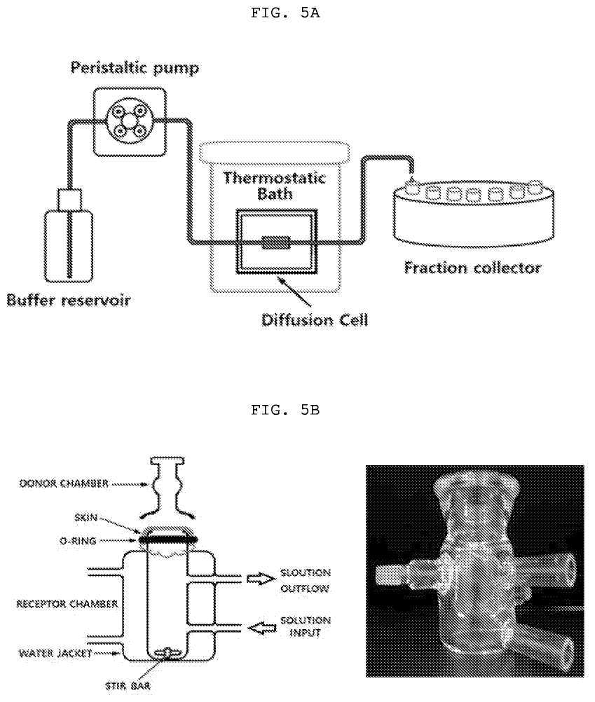

As a method of quantitatively testing a degree of penetration of the liquid chemical agent or the harmful toxic substance into the living skin, a diffusion cell (flow-through diffusion cell) test method using the mouse skin was performed. The test method followed “Guideline for in vitro Skin Absorption method, 2009 Dec. 10” established and reported by Ministry of Food and Drug Safety. However, the conditions were optimized for measuring the skin protection performance of the reactive topical skin protectant formulation of the present disclosure. The contents of the diffusion cell test method for testing the skin protection effect of the reactive topical skin protectant formulations of the present disclosure are shown in FIGS. 5A and 5B.

FIG. 5A schematically shows the summary of the diffusion cell (Flow-through diffusion cell) test method, and FIG. 5B schematizes the diffusion cell structure, in which the right side is a photograph of the diffusion cell used in the test of the present disclosure.

Penetration amounts of the applied liquid simulant which passed through the mouse skin, and diffused and moved into the buffer solution were added to a total of seven vials immediately after loading the simulant agent, after 2 hours, after 6 hours, after 10 hours, after 14 hours, after 18 hours, and after 22 hours. This was collected with a 0.2 μm syringe filter, filtered, and quantitatively analyzed with high performance liquid chromatography (HPLC).

The analysis conditions of HPLC were using a UV detector, and a mixture of HPLC grade methanol and tertiary distilled water at a ratio of 55:45 (v/v) as a mobile phase. In the analysis, C8 (4.6×100 mm, 5 μm) was used as a column, the temperature of the column was 35° C., a flow rate was 1.0 ml/min, and an injection amount was 10 μl.

The test results were interpreted as an average value obtained by repeating the diffusion cell test using the mouse skin under the same conditions three times, and the penetration amount of the liquid chemical agent or the harmful toxic substance into the skin was calculated using the following Calculation Formula (1):

$${M}_{n} = {VC}_{n} ( t ) + \frac{{S}_{n}}{2} + {\sum }_{i = 1}^{n - 1} {S}_{i} \qquad ( 1 )$$

wherein Mₙ is the total amount of the sample penetrating into the skin (liquid chemical agent or harmful toxic substance), VCₙ(t) is the amount of a sample penetrating into the diffusion cell,

$${\sum }_{i = 1}^{n - 1} {S}_{i}$$

is the amount of the sample penetrating into the skin, received in a vial to the nth time point, and

$$\frac{{S}_{n}}{2}$$

is the amount of the sample penetrating to the nth time point.

Then, the protective rate was calculated using the following Calculation Formula (2):

$$Protective rate ( \% ) = \frac{[ Dose ( μ g ) - Penetration amount ( μ g )}{Dose ( μ g )} \times 100 \qquad ( 2 )$$

wherein dose (μg) is volume (ml)×agent density (g/cm³)×1000 of the agent administered on the surface of the mouse skin, and penetration amount (μg) is a value obtained by analyzing the sample obtained in each collection step by HPLC and following Calculation Formula (1).

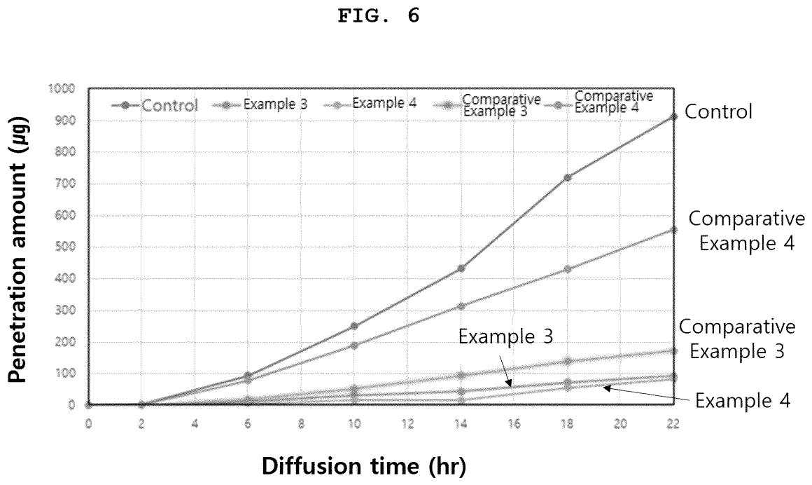

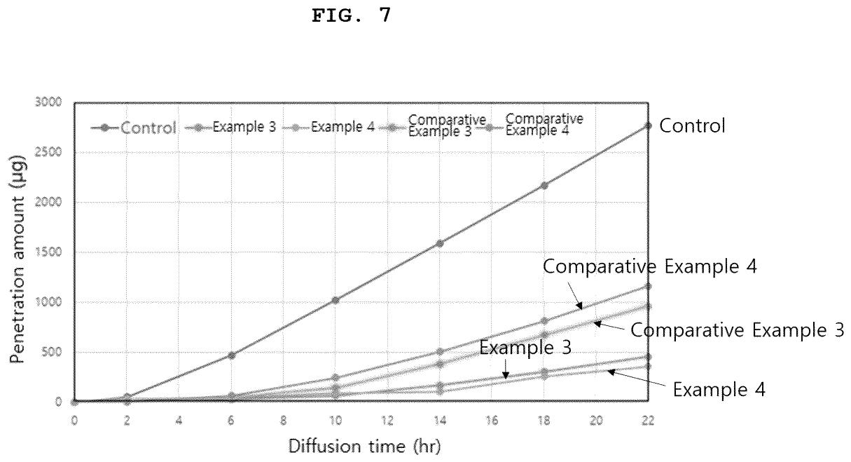

The diffusion cell test results performed as described above are shown in the following Tables 7 and 8 and FIGS. 6 and 7.

TABLE 7
Diffusion cell penetration test results of
2-CEPS (dropping amount = 20 μl/2 cm²)

| | | | | Comparative | Comparative |
|---|---|---|---|---|---|
| | Control | Example 3 | Example 4 | Example 3 | Example 4 |
| Diffusion | Penetration | Penetration | Penetration | Penetration | Penetration |
| time (hr) | amount (μg) | amount (μg) | amount (μg) | amount (μg) | amount (μg) |
| 0 | 0 | 0 | 0 | 0 | 0 |
| 2 | 6 | 0 | 0 | 0 | 4 |
| 6 | 186 | 22 | 12 | 34 | 158 |
| 10 | 500 | 62 | 30 | 104 | 378 |
| 14 | 864 | 86 | 30 | 186 | 626 |
| 18 | 1,440 | 144 | 110 | 278 | 860 |
| 22 | 1,824 | 184 | 166 | 344 | 1,110 |
| Protective | 92.23 | 99.22 | 99.29 | 98.53 | 95.27 |
| rate (%) | | | | | |

In Table 7, the protective rate (%) of each formulation was a value calculated by a total penetration amount (μg) by 22 hours for the dose (μg) of 23,480 μg since the volume of 2-CEPS administered with a micropipette on a mouse skin area of 2 cm² attached to the diffusion cell was 20 μl and a density of 2-CEPS was 1.174 g/cm³.

From the results of Table 7, it was found that the protective rate of the control without formulation against 2-CEPS was at a level of 92% after 22 hours, but actually, the protective rate as such may be lethal considering the medium lethal dose (LD50) of common liquid chemical agents.

However, in Examples 3 and 4 to which the formulations of the present disclosure were applied, the 2-CEPS protective rate were 99.22% and 99.29% after 22 hours, which were excellent as compared with the formulation of the comparative example. At this time, the cumulative penetration amount trend of 2-CEPS for each of the formulations penetrating the mouse skin of the diffusion cell is shown in FIG. 6.

For Dichlorvos also, the penetration amounts of the formulations of Examples 3 and 4 of the present disclosure and the formulations of Comparative Examples 3 and 4 were measured as the same diffusion cell test method. The results are shown in the following Table 8 and FIG. 7.

Likewise, the protective rate (%) of the formulation against Dichlorvos was also a value calculated by a total penetration amount (μg) by 22 hours for the dose (μg) of 28,400 μg since the volume of Dichlorvos administered with a micropipette on a mouse skin area of 2 cm² attached to the diffusion cell was 20 μl and a density of Dichlorvos was 1.420 g/cm³.

TABLE 8
Diffusion cell penetration test results of
Dichlorvos (dropping amount = 20 μl/2 cm²)

| | | | | Comparative | Comparative |
|---|---|---|---|---|---|
| | Control | Example 3 | Example 4 | Example 3 | Example 4 |
| Diffusion | Penetration | Penetration | Penetration | Penetration | Penetration |
| time (hr) | amount (μg) | amount (μg) | amount (μg) | amount (μg) | amount (μg) |
| 0 | 0 | 0 | 0 | 0 | 0 |
| 2 | 54 | 11 | 36 | 2 | 7 |
| 6 | 470 | 30 | 44 | 51 | 65 |
| 10 | 1,022 | 67 | 92 | 147 | 247 |
| 14 | 1,589 | 170 | 106 | 382 | 505 |
| 18 | 2,169 | 306 | 259 | 673 | 811 |
| 22 | 2,766 | 456 | 360 | 962 | 1,162 |
| Protective rate (%) | 90.26 | 98.39 | 98.73 | 96.61 | 95.91 |

From the results of Table 8, it was found that the protective rate of the control against Dichlorvos was at a level of 90% after 22 hours, but the protective rate as such may be lethal like 2-CEPS considering the half lethal dose (LD50) of a common liquid chemical agent.

However, in Examples 3 and 4 which were the reactive topical skin protectant formulations of the present disclosure, the protective rate against Dichlorvos were 98.39% and 98.73% after 22 hours, which were excellent as compared with the formulations of Comparative Examples 3 and 4. At this time, the cumulative penetration amount trend of Dichlorvos for the mouse skin is shown in FIG. 7.

The present disclosure may provide a new reactive topical skin protectant formulation which may safely protect the skin from various lesions and risks caused by penetration of liquid chemical agents or harmful toxic substances into the human skin.

What is claimed is:

1. Reactive topical skin protectant formulations comprising: water or moisture; plate-like inorganic particle powder; and one or more selected from the group consisting of a) liquid perfluoride, b) polyol, c) perfluorinated polymer powder, d) polysaccharide, e) liquid silicon-based material, and f) additives.

2. The reactive topical skin protectant formulations of claim 1, wherein the water or moisture is included at 0.001 to 2 wt % based on the total weight of the reactive topical skin protectant formulations.

3. The reactive topical skin protectant formulations of claim 1, wherein the plate-like inorganic particle powder is included at 0.1 to 30 wt % based on the total weight of the reactive topical skin protectant formulations.

4. The reactive topical skin protectant formulations of claim 1, wherein the moisture is included at 0.1 to 5 parts by weight with respect to 100 parts by weight of the plate-like inorganic particle powder.

5. The reactive topical skin protectant formulations of claim 1, wherein the plate-like inorganic particle powder has an average particle diameter of 0.1 to 10 μm.

6. The reactive topical skin protectant formulations of claim 1, wherein the plate-like inorganic particle powder is one or more selected from mica, pearl, talc, bentonite, boron nitride, graphite, and aluminum powder pressed thinly with a roller.

7. The reactive topical skin protectant formulations of claim 1, wherein the plate-like inorganic particle powder is surface-treated with one or more inorganic substances selected from magnesium oxide, magnesium hydroxide, magnesium carbonate, aluminum oxide, aluminum hydroxide, silica, hydrated silica, calcium oxide, calcium hydroxide, calcium carbonate, titanium dioxide, titanium hydroxide, iron oxide, iron hydroxide, zinc oxide, zinc hydroxide, zinc carbonate, zirconium oxide, zirconium hydroxide, cerium oxide, and cerium hydroxide.

8. The reactive topical skin protectant formulations of claim 7, wherein the surface treatment includes: dispersing the plate-like inorganic particle powder in a metallic salt aqueous solution; adding an alkaline aqueous solution to form precipitate in a range of pH 9 to 10 to perform aggregation; and performing heating at 400 to 500° C. for 30 minutes to 3 hours.

9. The reactive topical skin protectant formulations of claim 7, wherein the inorganic substance is included at 0.5 to 20 parts by weight with respect to 100 parts by weight of the plate-like inorganic particle powder.

10. The reactive topical skin protectant formulations of claim 1, wherein 10 to 85 wt % of the liquid perfluoride, 5 to 60 wt % of the polyol, 5 to 60 wt % of the perfluorinated polymer powder, 0.001 to 2 wt % of the polysaccharide, and 0.01 to 10 wt % of the additives are included based on the total weight of the reactive topical skin protectant formulations.

11. The reactive topical skin protectant formulations of claim 1, wherein the perfluoride is represented by the following Structural Formula 1: 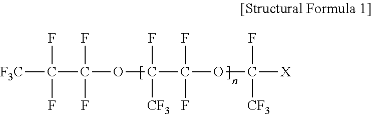
wherein n is an integer of 1 to 500, and X is selected from the group consisting of F, acyl fluoride, acrylate, C1-C4 alcohol, carboxylic acid, and methyl ester.

12. The reactive topical skin protectant formulations of claim 1, wherein the perfluorinated polymer powder has an average particle diameter of 1 to 10 μm and a specific surface area of 3 to 15 m²/g.

13. The reactive topical skin protectant formulations of claim 1, wherein the liquid silicon-based material is represented by the following Structural Formula 2: 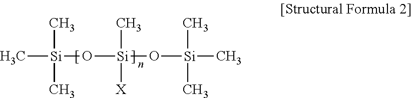
wherein n is an integer of 1 to 500, and X is hydrogen or a methyl group.

14. The reactive topical skin protectant formulations of claim 1, wherein the additive is one or more selected from a fluorine-based film forming agent, a fluorine-based surfactant, a fluorine-based thickener, a fluorine-based solvent, a silicon-based film forming agent, a silicon-based surfactant, a silicon-based thickener, a lipid-based raw material, and an inorganic salt adjusting agent.

15. The reactive topical skin protectant formulations of claim 1, wherein the reactive topical skin protectant formulations are formulated into one or more selected from the group consisting of cream, ointment, gel, foundation, and stick.

16. The reactive topical skin protectant formulations of claim 1, wherein the reactive topical skin protectant formulations are for protecting the skin from lesions caused by transdermal penetration of liquid chemical agents or harmful toxic substances in contact with the skin.
